(19) United States
(12) Patent Application Publication
(10) Pub. No.: US 2025/0314637 A1
(43) Pub. Date: Oct. 9, 2025

(54) HYDROGEL MICROENVIRONMENT FOR GASTRIC TISSUE CULTURE MODEL

(71) Applicant: THE ROYAL INSTITUTION FOR THE ADVANCEMENT OF LEARNING/MCGILL UNIVERSITY, Montréal (CA)

(72) Inventors: Joseph Matthew KINSELLA, Montréal (CA); Madison SANTOS, Montreal (CA); Ariel CORSANO, Toronto (CA); Isabelle DUMMER, Montreal (CA)

(21) Appl. No.: 19/169,512

(22) Filed: Apr. 3, 2025

Related U.S. Application Data
(60) Provisional application No. 63/575,240, filed on Apr. 5, 2024.

(51) Int. Cl. G01N 33/50; C12N 5/071; C12N 5/09

(52) U.S. Cl. CPC G01N 33/5011; C12N 5/0679; C12N 5/0693; C12N 2503/02; C12N 2513/00; C12N 2533/90

(57) ABSTRACT

There is provided a gastric tissue tumour model that is three-dimensional comprising from 0.8 to 1.2 w/v % alginate, from 1.7 to 2.3 w/v % gelatin and gastric decellularized extracellular matrix (dECM). The gastric tissue model is for example an esophageal cancer tumour model which advantageously replicates the tumour microenvironment for esophageal cancer and can be used to perform a three-dimensional culture of esophageal tumour cells or metastatic cells that often metastasize into the esophagus.

The present application claims the priority of U.S. provisional application No. 63/575,240 filed on Apr. 5, 2024 and incorporated herein by reference in its entirety.

### TECHNICAL FIELD

This disclosure relates to the field of tissue modeling, specifically in vitro tissue models or tumour models that are three-dimensional (3D) and allow to culture and test treatments against the tumours. Particularly, the present disclosure relates to a hydrogel that mimics the microenvironment of gastric tissues such as gastric tumours.

### BACKGROUND OF THE ART

Esophageal adenocarcinoma is a cancer caused by malignant gland cells forming a tumour that impedes typical function in the esophageal lining. This devastating disease is most often diagnosed in its advanced stages, at which point cancer cells will have spread throughout the esophagus or metastasized to other parts of the body. While treatment to mitigate symptoms is possible, it is rarely curable, with a five-year net survival rate of around 16%.

Most landmark discoveries in understanding cancer biology and advancing new therapeutic strategies have used in vitro monolayer cell culture systems or preclinical in vivo small animal models. While these models are beneficial, cell culture models lack the dimensional aspects of tumour growth, and tumour xenografts introduce non-native biological cues not found in humans to the models. New tumour models are needed to recapitulate the tumour microenvironment's (TME) architecture, heterogeneity, and bioactive components.

The poor prognosis, few therapeutic options for esophageal cancer, and the lack of appropriate tumour models hinder the clinical translation of therapies and sophisticated cancer studies. Therefore, it would be desirable to have a three-dimensional in vitro tumour model that recapitulates the esophageal cancer environment. More generally, it would be desired to have a tissue model that can not only be used to recapitulate the esophageal cancer environments, but also other gastric environments (that are cancerous or not).

### SUMMARY

In one aspect, there is provided a gastric tissue model that is three-dimensional comprising from 0.8 to 1.2 w/v % alginate, from 1.7 to 2.3 w/v % gelatin and gastric decellularized extracellular matrix (dECM). The gastric tissue model can be free of detergents. The gastric dECM is obtained by subjecting a gastric tissue to a decellularization (supercritical carbon dioxide decellularization, an enzymatic decellularization, detergent decellularization or a combination thereof). Preferably, the alginate is present in a concentration of from 0.9 to 1.1 w/v %, or about 1 w/v %. Preferably, the gelatin is present in a concentration of from 1.9 to 2.1 w/v %, or about 2 w/v %. The gastric tissue model is adapted to receive three-dimensional cell cultures. The gastric tissue model is itself three dimensional and can have a thickness of at least 200 μm. In some embodiments, the gastric dECM represents at least 95 wt. % of the gastric tissue model. In further embodiments, the gastric tissue model consists of the alginate, the gelatin and the dECM. The gastric tissue model is for example an esophageal tissue model, an upper gastric tissue model or a colon tissue model, and optionally an esophageal cancer tissue model.

In a further aspect, there is provided a method of producing the gastric tissue model that is three dimensional, the method comprising: a) performing a supercritical carbon dioxide decellularization on a gastric tissue to obtain a gastric decellularized extracellular matrix (dECM), and b) preparing a composite material by mixing the dECM with 0.8 to 1.2 w/v % alginate and 1.7 to 2.3 w/v % gelatin. Preferably, the alginate is present in a concentration of from 0.9 to 1.1 w/v %, or about 1 w/v %. Preferably, the gelatin is present in a concentration of from 1.9 to 2.1 w/v %, or about 2 w/v %. In some cases, the gastric tissue is porcine gastric tissue. In some embodiments, the tumour model has a thickness of at least 300 μm.

The gastric tissue model of the present disclosure can be used for screening drugs for the treatment of gastric conditions such as gastric cancer or more specifically esophageal cancer. This allows for the personalized treatment of a patient having a gastric condition, or for culturing a three-dimensional cell line (e.g. cancer cells, primary cells or immortalized cells). The cancer cell line is for example a metastatic cell line or an esophageal cancer cell line in the form of cell spheroids. As such, in a still further aspect, there is provided a method for screening drugs for the treatment of gastric cancer, said method comprising contacting the gastric tissue model as described herein and comprising gastric cancer cells with a candidate drug for treating the gastric cancer, and evaluating the efficacy of the candidate drug for treating said gastric cancer.

In another aspect, there is still provided a personalized treatment of a patient having a gastric condition such as a gastric cancer, said treatment comprising i) screening for drugs for the treatment of said gastric condition by contacting the gastric tissue model as described herein with candidate drugs for treating the gastric condition, and selecting a drug having efficacy in treating said gastric condition, and administering said drug having efficacy in treating said gastric condition to the patient.

Yet in another aspect, there is also provided a method for culturing a three-dimensional cell line, said method comprising seeding a cell line in the gastric tissue model as described herein. The initial cells can be provided as individual cells which form a three dimensional culture after seeding or the initial cells can be provided already as three dimensional (e.g. spheroid or organoid).

Many further features and combinations thereof concerning the present improvements will appear to those skilled in the art following a reading of the instant disclosure.

### DESCRIPTION OF THE DRAWINGS

FIG. 1A is a graph showing the absorbance measured indicating the sodium dodecyl sulfate percentage (square is the sample and circle are the calibration points).

FIG. 1B is a graph showing absorbance measured indicating the sDNA concentration (square is the sample and circle are the calibration points).

FIG. 2A is a graph showing the absorbance measured indicating the collagen content (square is the sample and circle are the calibration points).

FIG. 2B is a graph showing the absorbance measured indicating the glycosaminoglycan content (square is the sample and circle are the calibration points).

FIG. 3A is a graph showing the storage modulus and shear modulus in function of the shear strain.

FIG. 3B is a graph showing the storage modulus and shear modulus in function of time.

FIG. 4A is a graph showing the storage modulus and shear modulus in function of the shear strain comparing the A3G8dECM and A2.5G5dECM samples.

FIG. 4B is a graph showing the storage modulus and shear modulus in function of time comparing the A3G8dECM and A2.5G5dECM samples.

FIG. 5A is a graph showing the storage modulus and shear modulus in function of the shear strain for the sample A1G2dECM.

FIG. 5B is a graph showing the storage modulus and shear modulus in function of time for the sample A1G2dECM.

FIG. 5C is a graph showing the shear rate for the sample A1G2dECM.

FIG. 6 is a microscopy image showing the formation of a spheroid (circled on the image).

FIG. 7A is a graph showing the fluorescent intensity indicative of cell growth in function of time (days 0, 1, 2, 3 and 4) in the sample A1G2dECM.

FIG. 7B is a graph showing the fluorescent intensity indicative of cell growth in function of time (days 0, 1, 2, 3 and 4) in the control A1G2PBS.

FIG. 8 is a graph showing the relative intensity compared to day 0 as indicative of cell growth over time.

### DETAILED DESCRIPTION

There is provided a gastric tissue model which was created using a hydrogel formulation of about 1 w/v % alginate and about 2 w/v % gelatin with decellularized extracellular matrix (ECM) from gastric tissue. The gastric tissue model exhibits physiochemical, temporal, and mechanical properties similar to esophageal environment and has a wide cell-type compatibility for testing different tumours including metastasis. Although the present gastric tissue model was initially designed to mimic esophageal environment, gastric environments are all similar and therefore the model is applicable to gastric tissues generally. In some embodiments, the tumour model is an upper gastric tissue, a colon tissue, or an esophageal tissue. The present model improves on 3D cell growth by retaining tissue's native structural polymers, cell adhesion, and signalling pathways within the hydrogel structure. With complex composition, polymers of the ECM support cell spreading, migration, and transformation with improved in vivo fidelity. Temporally, the present gastric tissue model can support ECM remodeling during cell growth, which is a key event in cancer metastasis that is not represented in traditional tumour models. Finally, in preferred embodiments, the model mimics the Young's modulus of murine esophageal stromal tissue, which plays a significant role in defining the tumour microenvironment, directing metastasis, and determining drug and immunotherapy penetration into the tumour core.

The term “about” as used herein in the context of a concentration value means±20%, ±15%, ±10%, ±5% or ±3%.

Functionally, the present in vitro gastric tissue model exhibits shear thinning within the safe range for human cells which makes the hydrogel system compatible with extrusion bioprinting. This is a further advantage of the present model, which can therefore be bioprinted. Additionally, the in vitro gastric tissue model has constant gelation kinetics across operating temperatures which allows manipulation of the tumour models on the lab bench, and in an incubator without compromising the desirable properties outlined above which mimic the gastric environment and in some embodiments the esophageal tumour environment.

In embodiments where the cells are cancerous, a model capable of hosting such cancer cells allows for a mechanistic understanding of tumour formation while minimizing the need for in vivo animal models. It also provides the framework for more sophisticated in vitro models that represent the heterogeneity of tumours by introducing co-cultures and more complex 3D architectures. Applications of the present model include screening novel targeted drugs and immunotherapies for esophageal cancer and more sophisticated studies of how interactions between cancer cells and their environment influence tumour mechanics and malignant cell growth.

As presented above, recapturing the mechanical properties of the tumour microenvironment (TME) is essential in cancer models, as mechanical changes impact the ability of drugs to reach the tumour core. Although the reasoning is presented with regards to tumours, the relevance of properly mimicking a gastric tissue can also be used to model any other type of treatment or disease, particularly when it comes to the mechanical changes in three dimension. In contrast, traditional in vitro cell monolayer models lack the three-dimensional aspect of the tissue (e.g. TME). For example, in vivo small animal tumour xenografts introduce non-human factors into tumour growth. The gastric tissue model of the present disclosure is a 3D in vitro tissue model that mirrors the biomechanical and pathophysiological characteristics of gastric tissues, for example esophageal cancer. Decellularized extracellular matrix (dECM) was selected as a bioactive additive for the gastric models and was obtained by decellularization of porcine gastric tissue. Decellularization was validated by spectrophotometric assays, confirming the retention of structural proteins and glycosaminoglycans. This retention lent itself to dECM gelation at physiological temperature and a high yield point (119.61 Pa) when mechanically characterized by shear rheology. The compositional and structural material characterization was used to appropriately tune the material mechanical properties to imitate esophageal tissue mechanics (which are similar to gastric tissues in general). To this end, alginate-gelatin hydrogels of varying concentrations have been formulated. It was found that a model composite hydrogel comprising 1% (w/v) sodium alginate and 2% (w/v) gelatin in pure dECM (labeled A1G2dECM) effectively replicates the tumour microenvironment for esophageal cancer which is similar to other gastric tissue (or the same as upper gastric tissue and colon tissue). This shear-thinning formulation successfully replicated the Young's modulus of esophageal tissue (12.21+/−0.32 kPa) with temperature invariant properties within the range of operating temperatures which is ideal for bioprinting applications.

The dECM of the present gastric tissue model is biologically derived from animal gastric tissue. Although porcine tissue was what was used in the Example section below, other animals or human sources for the gastric tissue, for example esophageal tissue can be used. The biomolecular environment obtained from porcine gastric tissue was selected for the particular embodiment of esophageal cancer, because it has similar properties to the esophageal tumour microenvironment. The ECM is a complex, tissue-specific structure composed of various macromolecules. The mechanical properties of this structure induce cellular responses, which contribute to the development and differentiation of cells. Additionally, ECM reorganization is critical in modulating tumour mechanical properties, such as stiffness and migration, which promote tumour growth, metastasis, and drug resistance. Decellularized ECM (dECM) molecules are extracted from tissues and formulated into a gel to incorporate this complex environment into the model. dECM provides bioactive properties to the model, including promoting cell proliferation and cell-cell interactions.

The dECM can be obtained using a solvent-free decellularization protocol using supercritical carbon dioxide (scCO₂) which is a non-toxic alternative to traditional decellularization, which could offer faster treatment and reduce damage to the dECM. ScCO₂ has desirable solvent properties within a physiologic temperature range, making it an attractive alternative. The current mechanism behind scCO₂ decellularization is not completely known. The two current hypotheses are as follows: the cells burst through a combination of the supercritical environment and the pressure, and as the system depressurizes, cellular material exits the ECM; supercritical extraction removes the cellular material directly.

dECM was selected as a bioactive additive to the formulation due to the innate conservation of native extracellular matrix structural and chemical factors that enhance cell proliferation through the retention of native pathways. In embodiments where the gastric tissue model is an esophageal cancer model, dECM also facilitates cancer tumour model fidelity to in vivo tumours by providing the framework for the eventual use of patient esophageal tissues in developing these models. By developing a direct pathway to tumour modelling with patient-derived cell cultures on human esophageal matrices, these models are expected to exhibit significant conservation of in vivo tumour characteristics which will positively contribute to the accuracy of their behaviour.

Since the dECM can be obtained by a solvent-free scCO₂ decellularization, in a preferred embodiment, the gastric tissue model is free of detergents such as sodium dodecyl sulfate (SDS) and Triton™ X-100 (C₈H₁₇C₆H₄(OC₂H₄)₉₋₁₀OH, Cas No. 9002-93-1).

Alternatively, although not the preferred embodiment, the decellularization can be performed enzymatically. Enzymatic methods for decellularization are generally performed with SDS and Triton™ X-100 detergents. Ionic detergents, like SDS, permeabilizes the cell membrane, which leads to cell removal, and separates the DNA. However, ionic detergents denature proteins and interfere with glycosaminoglycans (GAGs) which can negatively impact the dECM. This is one of the reasons supercritical carbon dioxide decellularization is preferred. Non-ionic detergents, such as Triton™ X-100, tend to be gentler than ionic detergents, as they only break lipid-lipid and lipid-protein interactions. However, these protocols result in low dECM yields that require significant purification and sterilization. Additionally, these protocols can be very time-consuming since they require several washes to remove residual detergents.

As stated above, the dECM is combined with alginate and gelatin to form the gastric tissue model. Mimicking the mechanical properties of the in vivo gastric tissue is important for developing accurate three dimensional models. In cancer applications, replicating the TME is essential in cancer models, as mechanical changes impact the ability of drugs to reach the tumour core. The motivation for the addition of alginate and gelatin is two-fold. First, to modulate the mechanical properties, namely stiffness, to recapitulate the physiological gastric environment (e.g. esophageal tumour environment). Secondly, to control the gelation kinetics to obtain an extrudable bioink capable of cell encapsulation. The advantages of a bioprinted model include control over the geometry and cell density of the 3D model and an additive manufacturing process suitable for parallel experimentation. Accordingly, in cancer modelling, the present gastric tumour model is a composite dECM gel formulation that is mechanically stable and has stiffness comparable to the intratumoral stroma.

The present disclosure provides a gastric tissue model that is capable of hosting cells such as cancer cells, immortalized cells or primary cells which allow for a mechanistic understanding of cell evolution and response to various stimuli or molecules. For example, the present gastric tissue model is well suited to model tumour formation while minimizing the need for in vivo animal models. Applications of this model include screening targeted drugs and immunotherapies for various gastric conditions such as esophageal cancer and more sophisticated studies of how interactions between cells and their environment influence disease. For example, in the case of cancer, the gastric tissue model can be used to study interactions between cells and their environment influence tumour mechanics and malignant cell growth.

Encapsulating patient-derived primary cells, immortalized cells or cancer cells into a bio-printable dECM gel can be used to proliferate and form spheroids, a marker of 3D growth and maturation. The present gastric tissue model can host such spheroids and have drugs tested specifically on the seeded cells from the patient. This is particularly advantageous for cancer as the present gastric cancer model allows to test for the specific heterogeneity of esophageal cancer that that patient has. In other words, personalized therapy is enabled by the present gastric tissue model. As shown in the Example section below using esophageal cancer as a proof of concept, the present gastric tissue model has demonstrated both cell proliferation and viability which are crucial to establish reliable models.

The gastric tissue model, in some embodiments, is defined as comprising from 0.8 to 1.2 w/v % alginate, from 1.7 to 2.3 w/v % gelatin and gastric dECM. In other embodiments, the gastric tissue model can be defined as consisting of 0.8 to 1.2 w/v % alginate, 1.7 to 2.3 w/v % gelatin and gastric dECM. The concentration of alginate can range from 0.85 to 1.15 w/v %, from 0.9 to 1.1 w/v %, or from 0.95 to 1.05 w/v %. The concentration of gelatin can range from 1.75 to 2.25 w/v %, from 1.8 to 2.2 w/v %, from 1.85 to 2.15 w/v %, from 1.9 to 2.1 w/v %, or from 1.95 to 2.05 w/v %. The alginate can be provided as sodium alginate, calcium alginate, and/or barium alginate.

The gastric tissue model of the present disclosure is a 3D culture system. A “3D culture system” can be define as a culture system adapted to receive three-dimensional cell structures such as cell aggregates, cell spheroids and organoids. The gastric tissue model is a hydrogel composite with a porosity adapted to receive the growth of cell spheroids, aggregates and organoids. The pores can therefore have a diameter that varies between 50 μm and 300 μm. The gastric tissue model is also considered three dimensional in the sense that the composite material, in some embodiments, has a thickness of at most 300 μm, 400 μm or 500 μm. In some embodiments, the thickness is from 200 to 500 μm, from 200 to 750 μm, from 200 μm to 1 mm. The gastric tissue model can also be characterized by a diameter or length of 1 to 10 mm.

### EXAMPLE

Porcine gastric tissue was obtained from two stomachs that were purchased from a commercial butcher. The upper halves of the stomachs were resected, and both were used to create a single batch of gel. The enzymatic decellularization was performed according to the protocol described in Kort-Mascort, Jacqueline, et al. (“Decellularized extracellular matrix composite hydrogel bioinks for the development of 3D bioprinted head and neck in vitro tumor models. ACS Biomaterials Science & Engineering 7.11 (2021): 5288-5300).

The dECM obtained after enzymatic decellularization was characterized using Picogreen™ and sodium dodecyl sulfate (SDS) assays. SDS plays an important role in decellularization by permeabilizing the cell membrane. However, SDS can frequently denature proteins and interfere with glycosaminoglycans (GAGs), thereby ruining the quality of the dECM. As such, an SDS removal step was performed once the cell walls have been permeabilized. To remove the SDS, the samples were strained and incubated in pure acetone at 4° C. overnight. The next day, the samples were separated into 50 mL centrifuge tubes, agitated with pure ice-cold acetone for 10 min, and centrifuged at 5000 g for 10 min at 4° C. The supernatant was discarded from the tube. This process was repeated 5 times. The success of the removal of SDS was investigated as follows.

A standard curve was created for the SDS detection assay with the following SDS percentages: 0.2, 0.1, 0.05, 0.025, 0.0125, 0.00625, 0.003125, and 0%. Then methylene blue (MB) was added at a 1:100 detergent:MB ratio. Then chloroform was added at a 1:2 ratio sample:chloroform. The solutions were then vortexed for one minute and then incubated for 10 minutes. The bottom layer was extracted and measured at an absorbance of 650 nm.

The Picogreen™ assay quantifies the nucleic acids remaining in the dECM. Decellularization removes all cellular material, including DNA, from the nucleus. The results for the enzymatic decellularization dECM should indicate low levels of nucleic acids. First, an aqueous working solution of dsDNA reagent was prepared by diluting the concentrated dimethylsulfoxide (DMSO) solution 200-fold in Tris-HCl and ethylenediaminetetraacetic acid (EDTA) buffer (TE). Then, a 2 μg/mL stock solution of dsDNA in TE was prepared. A standard curve was constructed with the following concentrations:

TABLE 1
Standard Curve Concentrations for Picogreen ™ Assay DNA

| Volume of | | Volume of 2 μg/mL | | Volume of diluted | Final DNA | |
|---|---|---|---|---|---|---|
| TE buffer | | DNA stock | | dsDNA reagent | concentration | |
| 0 | μL | 100 | μL | 100 μL | 1 | μg/mL |
| 90 | μL | 10 | μL | 100 μL | 100 | ng/mL |
| 99 | μL | 1 | μL | 100 μL | 10 | ng/mL |

| | | | | | |
|---|---|---|---|---|---|
| 100 | μL | 0 | μL | 100 μL | blank |

The volume of 100 μL of aqueous working solution of dsDNA Reagent was added to each well in a 96 well microplate reader plate. The plate was then mixed and incubated at room temperature for 2-5 minutes. The sample fluorescence was then measured using a fluorescence microplate reader. The excitation wavelength was 480 nm, and the emission wavelength was 520 nm. The measurements were made in triplicate.

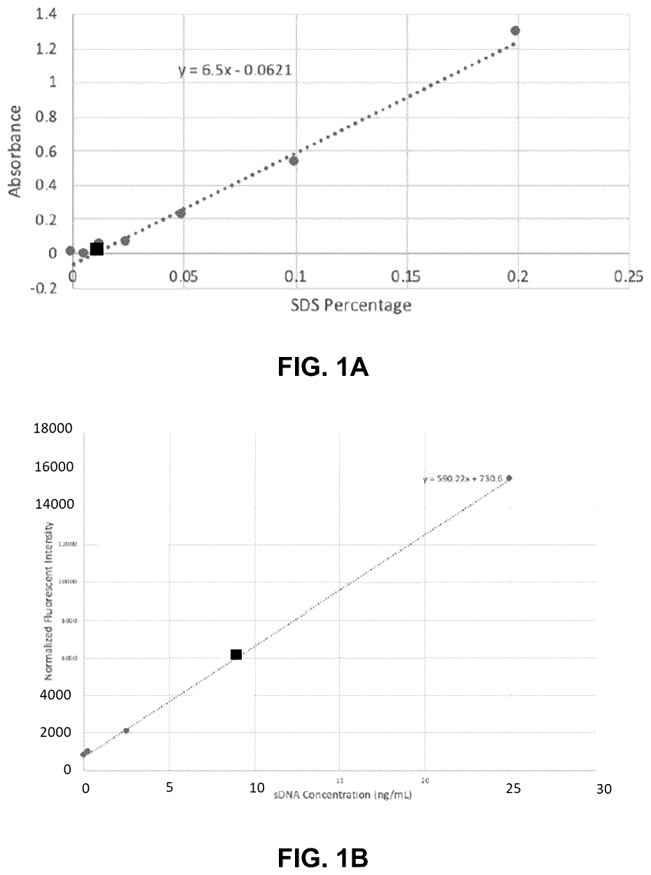

A standard curve was constructed for the Picogreen™ assay, displayed in FIG. 1A. Using the standard curve, the dsDNA concentration in the dECM was determined to be 9.05 ng/mL. This is lower than the DNA concentration in dECM used to make decellularized human lung bioink for 2D and 3D lung cell culture. However, since the purpose of the dECM is to mimic esophagus tissue, the measured dsDNA concentration is was good in that regard. As for the SDS assay, the SDS percentage of the dECM was 0.106% (FIG. 1B). This low value of SDS is desirable since SDS is an ionic detergent that denatures proteins and can interfere with GAGs, negatively impacting the dECM.

Two Biocolor™ assays were run: the sGAG (Blyscan™ sGAG assay standard kit) and the soluble collagen assays. GAGs are large complex carbohydrate molecules involved in certain proteins' regulations, such as growth factors and adhesion molecules. These molecules tend to be denatured during enzymatic decellularization. Collagen is the most abundant structural protein found in dECM. Since collagen content affects the mechanical properties of the composite gel, its quantification can provide insight into the gel formulation.

The sGAG assay was performed with the following values: 0, 1, 2, 3, 4, and 5 μg of GAG. Then 1 mL of dye reagent and 100 μL of enzymatic decellularization sample were added to each standard curve tube. After 30 minutes of incubation, the standard curve and sample tubes were centrifuged for 15 minutes at 13000 g. The supernatant was then decanted carefully, and 0.5 mL of dissociation reagent was added to each tube. The pellets formed during centrifugation were vortexed to release the bound dye. To remove bubbles, the tubes were centrifuged, after which the absorbances were measured at 656 nm. The measurements were made in triplicates.

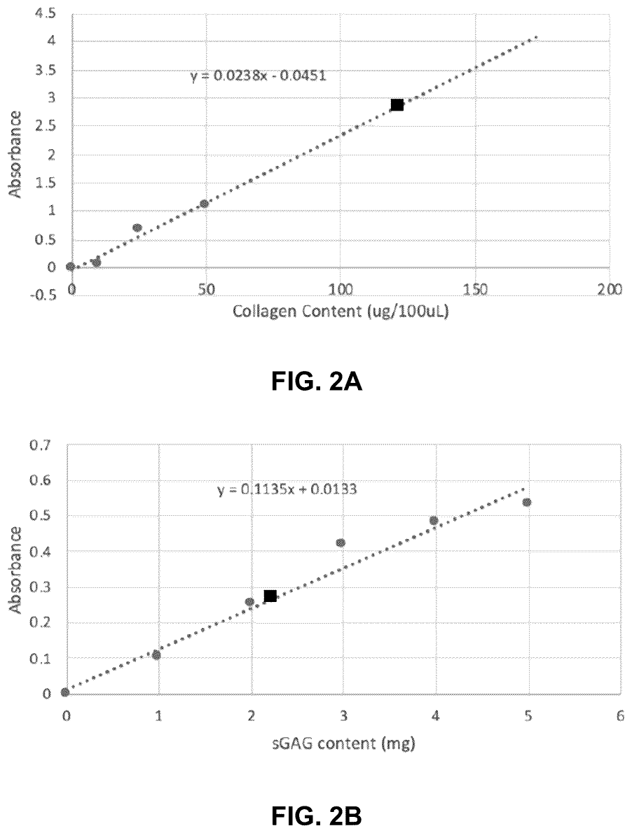

A standard curve was created for the soluble collagen assay with the following values: 0, 10, 25, and 50 μg of collagen. Then 1 mL of Sircol™ dye reagent was added to the standard curve and 100 μL of enzymatic decellularization sample. Each Eppendorf tube was centrifuged at 13000 g for 15 minutes after a 30-minute incubation period. The supernatant was then decanted, and 1 mL of alkali reagent was added. The pellets were then broken up to release the bound dyes into the solution, after which the absorbances were measured at 556 nm. The measurements were made in triplicates. The standard curve is displayed in FIG. 2A.

The standard curve for the sGAG assay is displayed in FIG. 2B. The quantity of sGAGs in 100 μL of the sample was found to be 2.2 μg using the standard curve, meaning a concentration of 2.2 μg/mL. This is tenfold higher than sGAG values for porcine tongue. Then using the collagen standard curve, the quantity of collagen in 100 μL of the sample was found to be 122.6 μg, for a concentration of 122.6 μg/mL. This value is relatively high, exceeding the standard curve range, which is a good sign. Rat stomach cardia was found to have a collagen content of 23.6%, a high value for non-ligament/non-skin tissue. This suggests that a dECM gel matrix should have a high collagen content to replicate the stomach properly.

To better understand the contributions of the enzymatically decellularized gastric tissue in a gel formulation, rheology was employed. The linear viscoelastic region (LVER) was determined by an amplitude sweep with a shear strain of 0.01% to 1000%, at a frequency of 0.1 Hz, at 37° C. To investigate temperature-dependent gelation, the dECM was incubated at 4° C. for 60 minutes, then the temperature was changed to 37° C. and incubated for another 90 minutes at 0.1% strain.

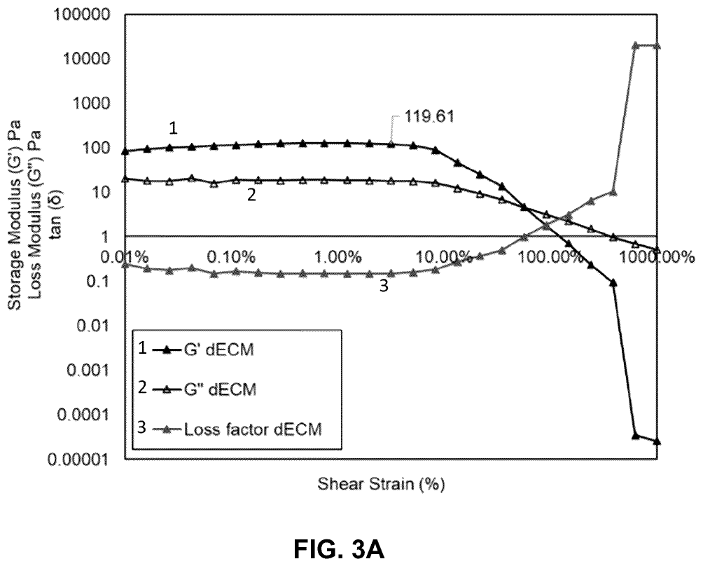

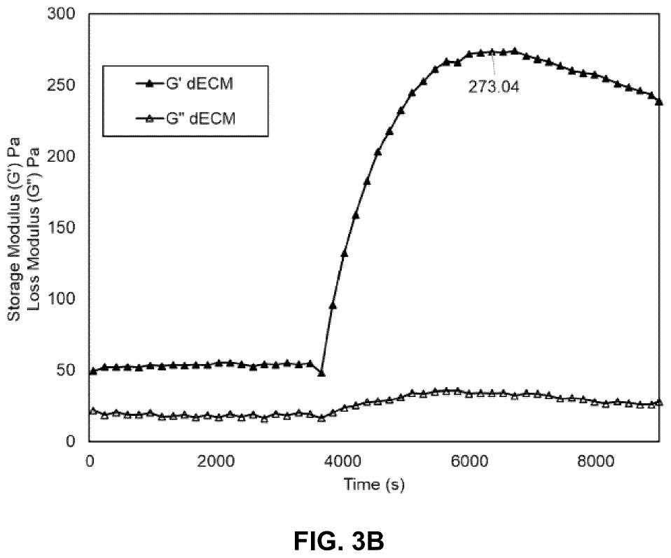

The amplitude sweep (FIG. 3A) revealed that the gastric dECM's yield point at 37° C. is 119.61 Pa, an order of magnitude larger than the reported value for dECM sourced from porcine tongue. A high yield point, in this case, is a desirable property because it indicates that fewer supporting materials, such as alginate and gelatin, would need to be added to enable bioprinting, thereby preserving a high concentration of dECM in the final gel formulation. The gelation kinetics analysis (FIG. 3B) indicated that the dECM behaved as a soft gel at 4° C. and reached its maximum storage modulus of 273.88 Pa at 37° C. and a tan(δ)=0.117.

Malignant transformation, tumour invasion, cancer metastasis, and chemotherapy delivery and resistance are all governed by the properties of the TME. Intratumoural stroma plays a large part in defining the TME, specifically within esophageal cancer. To develop a formulation that best simulates the mechanical properties of the esophageal TME (Young's modulus of 12.22 +/−3.05 kPa) a different decellularization method (ScCO₂ decellularization) was tested as well as different formulations (various concentrations of alginate and gelatin added). Supercritical decellularization was performed on the porcine gastric tissue for 4 hours at 37° C. and 2300 psi with 50 mL of 70% ethanol. The yield was around 90% and it should be noted that this yield is significantly higher than enzymatic decellularization. The yield obtained was around 5% of lyophilized dECM based on 500 g initial wet tissue which yielded 25.2 g of freeze dried dECM.

The rheological method and preparation of all samples are as follows. Before testing, the sample was cast in a 20 mm circular mold, and cross-linked by immersion in CaCl₂ for 30 s, followed by rinsing with phosphate buffered saline (PBS) 1X to ensure reproducibility and ease of handling. First, to determine each sample's linear viscoelastic region (LVER), an amplitude sweep was performed with a shear strain of 0.01% to 1000%, at a frequency of 0.1 Hz, at 24° C. To simulate bioprinting conditions, a gelation kinetics test was then explored. The composite bioinks were incubated at 37° C. for 60 minutes; then, the temperature was changed to 24° C. and incubated for another 90 minutes at 0.1% strain.

Two formulations were initially developed, A3G8dECM and A2.5G5dECM, specifically diversifying the proportions of gelatin and alginate to maximize the difference in the formulations. A3G8dECM were prepared by adding 3 w/v % sodium alginate and 8 w/v % gelatin to the ScCO₂ decellularized porcine tissue. A2.5G5dECM was prepared by adding 2.5 w/v % sodium alginate and 5 w/v % gelatin to the decellularized porcine tissue as described in described in Kort-Mascort, Jacqueline, et al. (supra).

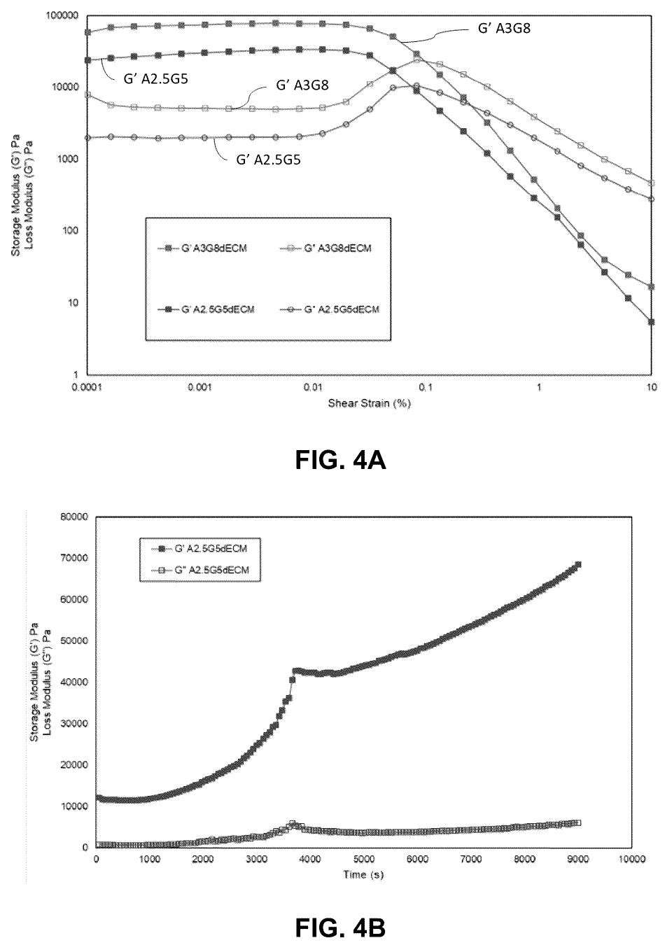

The amplitude sweep (FIG. 4A) revealed that the A3G8dECM bioink had an initial storage modulus of 58.90 kPa, which is well above the target Young's modulus of stromal tissue, and as such, was disqualified. Based on the amplitude sweep and gelation kinetics tests, the initial storage modulus of the A2.5G5dECM bioink under low strain (0.1%) ranged between 24.09 kPa at 24° C. and 12.17 kPa at 37° C. (FIG. 4B). A2.5G5dECM was close to the target goal of 12.22±3.05 kPa (deviating at the temperature of 37° C.). The gelation kinetics for A2.5G5dECM, however, disqualified it as a candidate to mimic esophageal on the basis of temperature stiffening behavior that was not conducive to maintaining the desirable mechanical properties throughout spheroid growth of tumours (FIG. 4B).

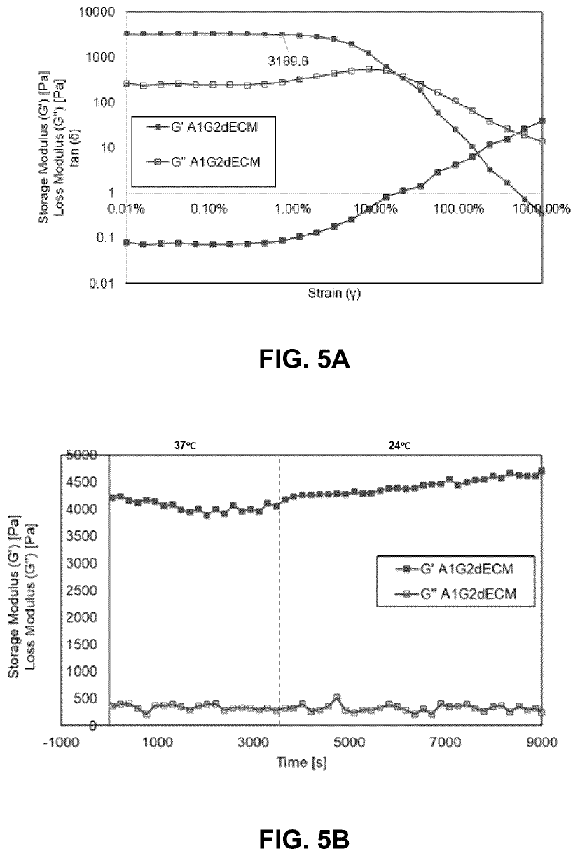

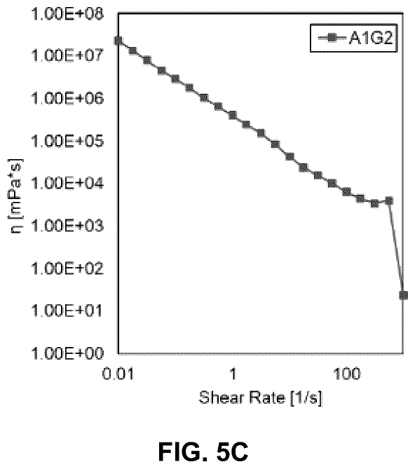

Following this, another formulation was developed, namely A1G2dECM by adding 1 w/v % sodium alginate and 2 w/v % gelatin to the decellularized porcine tissue as described in described in Kort-Mascort, Jacqueline, et al. (supra). The Young's modulus was calculated according to equation 1 (see below) and was found to effectively replicate the Young's modulus of murine esophageal stromal tissue at 12.21±0.32 kPa. The yield point (G′y=3.169 kPa) was well above the pressures used in bioprinting, confirming that bioprinting would occur within the LVER, and not cause permanent deformation to the gel (FIG. 5A). The gel was also observed to be shear thinning at a high shear rate, which is necessary for extrusion bioprinting (FIG. 5C). Through a trade-off between the inverse temperature gelation trends of gelatin and dECM, this formulation exploited the appropriate ratios of each to obtain temperature invariant gelation kinetics throughout all operating temperatures (FIG. 5B). This is vital for maintaining a physiologically accurate Young's modulus (E) throughout manipulations in the biological safety cabinet, and cell growth within the incubator.

$$Equation 1$$

$$( where v is the Poisson ’ s ratio )$$

$$E = 2 \times {( {G}^{′ 2} + {G}^{″ 2} )}^{0.5} \times ( 1 + v )$$

Owing to its desirable Young's modulus, shear thinning properties, and temperature invariant gelation kinetics between 25-37° C., A1G2dECM was shown to be a successful replication of the Young's modulus of murine esophageal stromal tissue.

MDA-MB-231 triple-negative breast cancer cells were selected as a cell line to learn adherent and 3D cell culture techniques and serve as a proof-of-concept for viability and spheroid formation in alginate-gelatin hydrogels. Two different gel models, A1G5 and A1G7, were chosen to compare the effect of gel formulation on cell growth and viability. A1G5 is a gel with 1 w/v % sodium alginate and 5 w/v % gelatin and A1G7 is a gel with 1 w/v % sodium alginate and 7 w/v % gelatin. A1G5 and A1G7 were selected because they have similar apparent Young's moduli, 7.79±1.88 kPa and 7.92±1.79 kPa, respectively, and can host MDA-MB-231 cells for 28 days. Additionally, A1G7 has been shown to promote larger and more rapid spheroid formation than A1G5 due to its larger ratio of cell-adhesive gelatin molecules. It was hypothesized that both models will be able to maintain cell viability greater than 80% over 15 days and that the A1G7 model will display greater spheroid formation.

MDA-MB-231 adherent cell culture was maintained in Dulbecco's Modified Eagle Medium (DMEM) cell culture media, supplemented with 10% fetal bovine serum (FBS) and 1% Penicillin-Streptomycin until it reached confluency, at which point cells were passaged. To transition adherent MDA-MB-231 cells from culture plates to 3D culture, the cells were mixed in A1G5 and A1G7 gels at a concentration of 1×10⁶ cells/mL. Then, the mixture was manually extruded through a 24-gauge nozzle into small droplets. The gels were crosslinked with CaCl₂ and washed them with PBS before transferring them to cell culture media. The media was changed every few days for two weeks. On day 8 and day 15 of 3D culture, the viability and growth of the cells were analyzed using live-dead staining under confocal microscopy. Cell counting was conducted using ImageJ™, and live and dead cell counts on day 15 are presented in Table 2.

TABLE 2
Live and dead cell counts on day 15 for A1G7 and A1G5

| Gel | Live | Dead |
|---|---|---|
| A1G5 | 837 | 260 |
| A1G7 | 1406 | 632 |

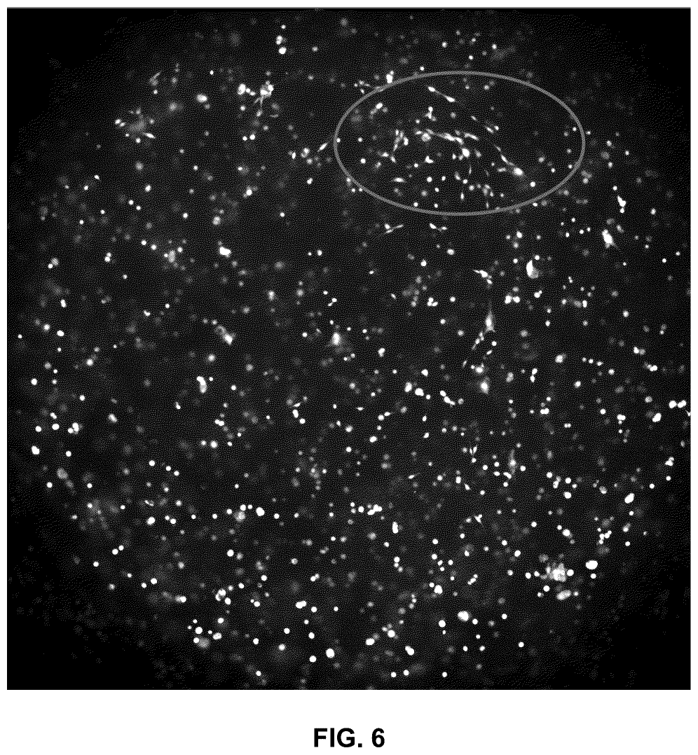

In the A1G7 model, the cell viability was 69%, lower than expected; however, this could be because the model does not have dECM components (and was only the gels) which support cell growth. Another reason could be contamination of the samples. In the A1G5 model, the cell viability was 76%. While this is higher than the A1G7 model, it is still lower than expected and not fully representative of the model, as only a partial cell count could be conducted since the whole z-stack of the model was not imaged. Early spheroid formation was observed on day 15 in A1G7 models, as seen in FIG. 6, but not in A1G5 models, confirming the hypothesis that the A1G7 models promote spheroid formation.

IMR-90 is a human fetal lung fibroblast cell line used as model fibroblasts in a wide variety of studies. This cell line was selected because the lung is a common metastatic site for esophageal cancer, and fibroblasts play an important role in synthesizing and remodeling the ECM. Additionally, the expression of the mCherry™ fluorescent marker allows for the analysis of cell growth by monitoring the fluorescent intensity. This experiment evaluates whether the optimized A1G2dECM gel formulation could host cell culture and maintain cell viability. A1G2PBS was also tested to compare the effect of dECM proteins on cell growth. It was hypothesized that IMR-90 cells encapsulated in A1G2dECM will have a greater cell growth rate than those in A1G2PBS gels.

IMR-90-mCherry™ cell culture was maintained, passaged, and encapsulated in A1G2dECM and A1G2PBS gels following the same procedure as the MDA-MB-231 experiment. 3D models were transferred to a 96-well plate in culture media, and the fluorescent intensity was measured with excitation and emission spectra of 587 nm and 610 nm, respectively, representing the peaks of mCherry™. Wells with cell culture media only served as a negative control. Five models of each gel were recorded for reproducibility, and the fluorescent intensity was measured over four days. The change in fluorescent intensity over time was used to represent the growth rate of the cells.

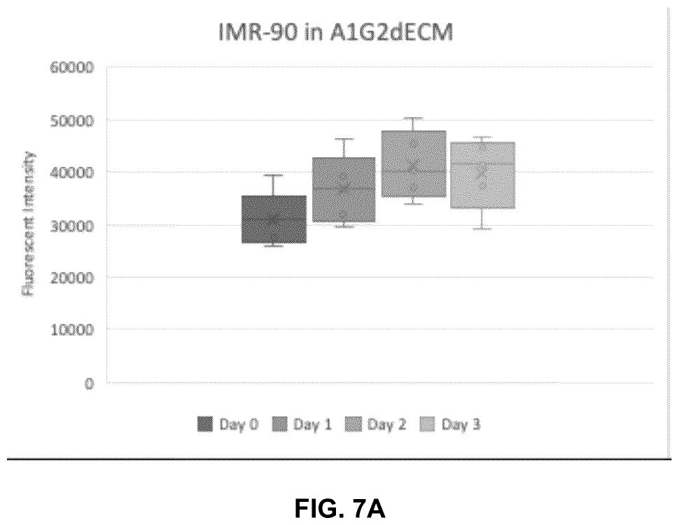

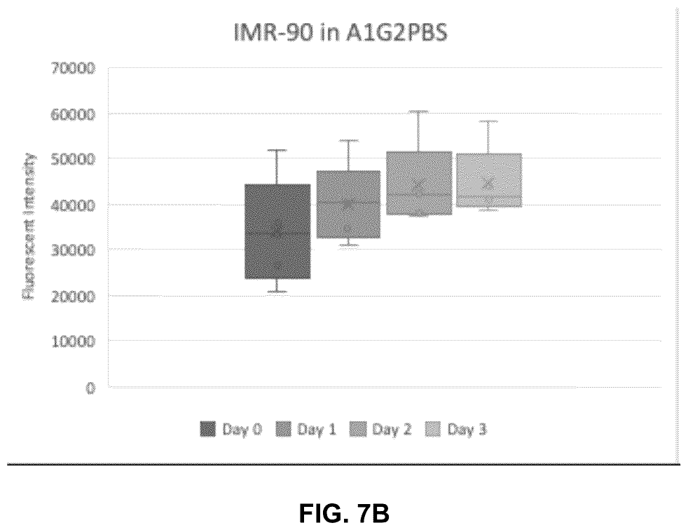

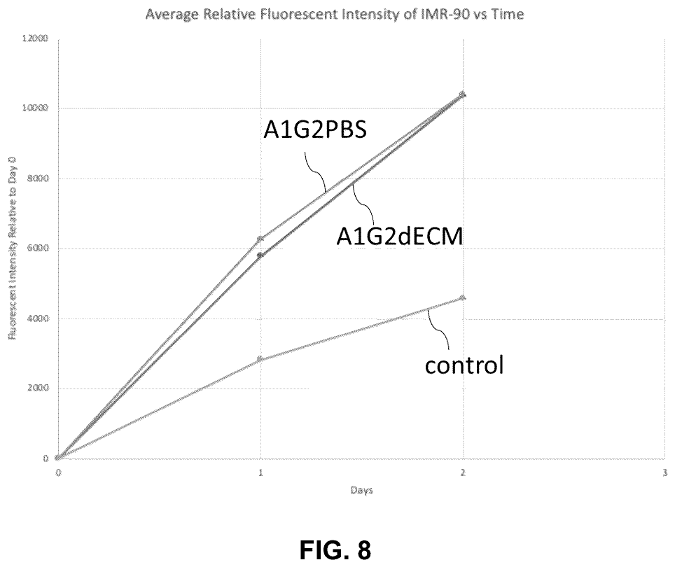

FIGS. 7A-7B show the fluorescent intensity distribution between samples and the average trend over four days. Both the A1G2dECM and A1G2PBS models show a trend of increasing intensity over time, indicating cell growth. The A1G2PBS samples have a larger variability, but neither set of samples contained outliers, which can be interpreted as each model having an approximately equal cell count. Both A1G2dECM and A1G2PBS trends showed a plateau or decrease in intensity on day 3, possibly due to a lack of available nutrients in the media since it was not changed. FIG. 8 compares the average fluorescent intensity over days 0-2 between the cell-laden A1G2dECM models, A1G2PBS models, and the control of cell culture media without cells. The trend in all groups is linear; surprisingly, the control group also displays a linear increase, likely because the gain for the readings was optimized based on the measurement from one of the control samples, which slightly increased between reads. The slope of A1G2dECM and A1G2PBS is very similar, indicating that the cell growth rate does not significantly vary in samples containing dECM proteins. Accordingly, it was shown that the optimized A1G2dECM gel can host 3D cell culture over four days.

What is claimed is:

1. An gastric tissue model that is three-dimensional comprising from 0.8 to 1.2 w/v % alginate, from 1.7 to 2.3 w/v % gelatin and gastric decellularized extracellular matrix (dECM).

2. The gastric tissue model of claim 1, being free of detergents.

3. The gastric tissue model of claim 1, wherein the dECM is obtained by subjecting a gastric tissue to a supercritical carbon dioxide decellularization, an enzymatic decellularization, detergent decellularization, or a combination thereof.

4. The gastric tissue model of claim 1, wherein the alginate is present in a concentration of from 0.9 to 1.1 w/v %.

5. The gastric tissue model of claim 1, wherein the gelatin is present in a concentration of from 1.9 to 2.1 w/v %.

6. The gastric tissue model of claim 1, wherein the gastric tissue model is adapted to receive three-dimensional cell cultures.

7. The gastric tissue model of claim 1, wherein the gastric tissue model has a thickness of at least 200 μm.

8. The gastric tissue model of claim 1, wherein the gastric tissue model is an esophageal tissue model, an upper gastric tissue model or a colon tissue model.

9. The gastric tissue model of claim 1, wherein gastric tissue model consists of the alginate, the gelatin and the dECM.

10. A method of producing an gastric tissue model that is three-dimensional, the method comprising: a) performing a supercritical carbon dioxide decellularization on a gastric tissue to obtain a gastric decellularized extracellular matrix (dECM), and b) preparing a composite material by mixing the dECM with 0.8 to 1.2 w/v % alginate and 1.7 to 2.3 w/v % gelatin.

11. The method of claim 10, wherein the alginate is provided in a concentration of from 0.9 to 1.1 w/v %.

12. The method of claim 10, wherein the gelatin is provided in a concentration of from 1.9 to 2.1 w/v %.

13. The method of claim 10, wherein the alginate is provided in a concentration of about 1 w/v %.

14. The method of claim 10, wherein the gelatin is provided in a concentration of about 2 w/v %.

15. The method of claim 10, wherein the gastric tissue is porcine gastric tissue.

16. The method of claim 10, wherein the gastric tissue model has a thickness of at least 200 μm.

17. A method for screening drugs for the treatment of gastric cancer, said method comprising contacting the gastric tissue model as defined in claim 1 comprising gastric cancer cells, with a candidate drug for treating the gastric cancer, and evaluating the efficacy of the candidate drug for treating said gastric cancer.

18. A personalized treatment of a patient having gastric cancer, said treatment comprising i) screening for drugs for the treatment of gastric cancer by contacting the gastric tissue model as defined in claim 1 comprising gastric cancer cells with candidate drugs for treating the gastric cancer, and selecting a drug having efficacy in treating said gastric cancer, and administering said drug having efficacy in treating said gastric cancer to the patient.

19. A method for culturing a three-dimensional cell line, said method comprising seeding a cancer cell line in the gastric tissue model as defined in claim 1.

20. The method of claim 19, wherein the cell line is a metastatic cell line or an esophageal cancer cell line in the form of cell spheroids.
